(19) United States
(12) Patent Application Publication
(10) Pub. No.: US 2023/0109161 A1
(43) Pub. Date: Apr. 6, 2023

(54) SYSTEMS AND METHODS FOR REDUCING SPASTICITY AFTER NEUROLOGICAL INJURY

(71) Applicant: Battelle Memorial Institute, Columbus, OH (US)

(72) Inventors: Eric C. Meyers, Columbus, OH (US); Michael Darrow, Missouri City, TX (US); Lauren Wengerd, Columbus, OH (US); Patrick Ganzer, Columbus, OH (US)

(21) Appl. No.: 17/944,539

(22) Filed: Sep. 14, 2022

Related U.S. Application Data
(60) Provisional application No. 63/251,894, filed on Oct. 4, 2021.

(51) Int. Cl. A61B 5/395; A61N 1/36

(52) U.S. Cl. CPC A61B 5/395; A61N 1/36031

(57) ABSTRACT

In a method of performing spinal reflex conditioning for an anatomical limb of a person, a spinal reflex is evoked by electrically stimulating a peripheral nerve of the anatomical limb, for example using stimulation electrodes disposed on an armband or leg band. The resulting spinal reflex is measured using electromyography (EMG) signals acquired from the anatomical limb. Vagus nerve stimulation (VNS) is performed in response to the measured spinal reflex satisfying a positive reinforcement criterion. The EMG may be high density EMG (HD-EMG) measured using a sleeve with a high density array of electrodes (e.g., at least 100 electrodes in an arm sleeve).

### CROSS-REFERENCE TO RELATED APPLICATION

This application claims priority to U.S. Provisional Patent Application Ser. No. 63/251,894 filed Oct. 4, 2021, the entire disclosure of which is incorporated by reference in its entirety herein.

### BACKGROUND

The following relates to the neurological injury rehabilitation arts, to methods and apparatuses for aiding stroke recovery, methods and apparatuses for aiding spinal cord injury recovery, and to the like.

Spasticity is a condition in which the muscles involuntarily tighten, thus preventing normal movement. Dysfunctional stretch reflexes underlie spasticity, and the condition can severely impair function of lower and/or upper limbs after a neurological injury such as a stroke or a spinal cord injury. For example, the stretching of a muscle can produce an afferent signal that feeds back to the peripheral nervous system, which in response generates an efferent signal to reinforce the muscle movement. A neurophysiological technique called the Hoffmann's Reflex (H-reflex) involves electrical stimulation of a peripheral nerve to electrically simulate the stretch reflex and thereby evoke a spinal reflex. In stroke patients with spasticity, the H-reflex (and similarly the stretch reflexes) of affected muscles are commonly higher than normal.

### BRIEF SUMMARY

In accordance with some illustrative embodiments disclosed herein, a method is disclosed of performing spinal reflex conditioning for an anatomical limb of a person. The method includes: evoking a spinal reflex by electrically stimulating a peripheral nerve of the anatomical limb; measuring the spinal reflex using electromyography (EMG) signals acquired from the anatomical limb; and performing vagus nerve stimulation (VNS) in response to the measured spinal reflex satisfying a positive reinforcement criterion.

In accordance with some illustrative embodiments disclosed herein, a system is disclosed for performing spinal reflex conditioning for an anatomical limb of a person. The system includes: an armband or leg band wearable on the anatomical limb and including electrodes arranged on the armband or leg band to electrically stimulate a peripheral nerve of the anatomical limb to evoke a spinal reflex; a sleeve wearable on the anatomical limb and including electrodes arranged to acquire EMG signals from the anatomical limb; a vagus nerve stimulation (VNS) device; and an electronic controller configured to evoke a spinal reflex by electrically stimulating the peripheral nerve of the anatomical limb using the armband or leg band, measure the spinal reflex using the sleeve, and perform VNS using the VNS device in response to the measured spinal reflex satisfying a positive reinforcement criterion.

In accordance with some illustrative embodiments disclosed herein, a non-transitory storage medium stores instructions readable and executable by an electronic processor to perform spinal reflex conditioning for an anatomical limb of a person by operations including: evoking a spinal reflex by controlling stimulation electrodes to electrically stimulate a peripheral nerve of the anatomical limb; measuring the spinal reflex using electromyography (EMG) signals acquired from the anatomical limb using electrodes of a sleeve configured to be worn on the limb; determining whether the measured spinal reflex satisfies a positive reinforcement criterion; and controlling a vagus nerve stimulation (VNS) device to deliver VNS to a vagus nerve of the person in response to the measured spinal reflex satisfying the reinforcement criterion.

In accordance with some illustrative embodiments disclosed herein, a method of performing spinal reflex conditioning for an anatomical limb of a person is disclosed. A spinal reflex is evoked by electrically stimulating a peripheral nerve of the anatomical limb, for example using stimulation electrodes disposed on an armband or leg band. The resulting spinal reflex is measured using electromyography (EMG) signals acquired from the anatomical limb. Vagus nerve stimulation (VNS) is performed in response to the measured spinal reflex satisfying a positive reinforcement criterion. The EMG may be high density EMG (HD-EMG) measured using a sleeve with a high density array of electrodes (e.g., at least 100 electrodes in an arm sleeve), although use of a lower density EMG, e.g. a single EMG electrode on the target muscle, is also contemplated.

### BRIEF DESCRIPTION OF THE DRAWINGS

Any quantitative dimensions shown in the drawing are to be understood as non-limiting illustrative examples. Unless otherwise indicated, the drawings are not to scale; if any aspect of the drawings is indicated as being to scale, the illustrated scale is to be understood as non-limiting illustrative example.

FIG. 1 diagrammatically shows a system for performing spinal reflex conditioning for an anatomical limb of a person.

FIG. 2 diagrammatically shows a method for performing spinal reflex conditioning for an anatomical limb of a person.

### DETAILED DESCRIPTION

An H-reflex can be evoked by electrically stimulating a peripheral nerve of an anatomical limb (arm or leg). A stretch reflex is evoked by muscle contractions. These types of reflexes are referred to herein as spinal reflexes. In a spinal reflex, an afferent signal is transmitted into the spinal cord due to the electrical stimulation of the peripheral nerve (in an H-reflex) or by muscle contraction (in a stretch reflex), and the afferent signal arriving at the spinal cord or other upper region of the peripheral nerve system triggers an efferent signal sent to the muscle. Such a spinal reflex is not volitionally controlled, and may involve only the peripheral nervous system (e.g., the spinal cord). Spinal reflexes are normal physiological responses that can beneficially reinforce motor actions to produce smooth and well controlled motor actions.

If the spinal reflex is insufficient for desired motor activity, this is referred to as hyporeflexia. A hyporeflexia response can manifest clinically as a weakness in muscles such as weak leg muscles that can cause falls (although there can be additional or other underlying physiological conditions leading to this condition), in which the motor action is incomplete or inadequate.

On the other hand, hyperreflexia is the situation in which the spinal reflex is too strong for the desired motor activity. Hyperreflexia commonly manifests as muscle spasticity, and is a common condition in stroke and spinal cord injury (SCI) patients. The spasticity can significantly degrade motor control.

In H-reflex conditioning, the stretch reflex of a certain skeletal muscle is conditioned using reinforcement based on the amplitude of the resulting reflexive muscle activity. Patch electrodes can be placed over the target muscles (such as the soleus muscle in the leg) to monitor spasticity which corresponds to the H-reflex. In one approach, the feedback can be visual, such as a computer display indicating whether spasticity is detected.

However, this example has certain deficiencies. The patch electrodes may not detect spasticity in muscles that are not monitored by the patch electrodes. The visual feedback involves higher cognitive centers, e.g. the visual cortex along with conscious recognition of the visual feedback and association to the spasticity or lack thereof. Since the spinal reflex is a nonvolitional reflex response, such positive feedback to the higher cognitive centers may not be effective for conditioning the spinal reflex. Still further, use of electrode patches and visual feedback typically entails a dedicated, stationary therapy setup, thus limiting the times and places where the patient can engage in the spinal reflex conditioning.

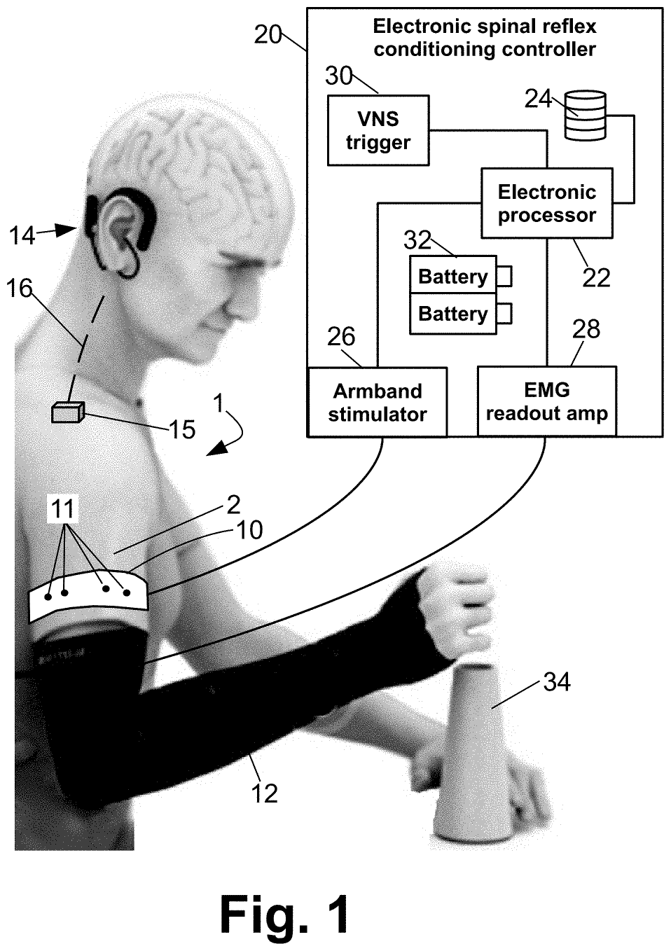

With reference to FIG. 1, an illustrative system is diagrammatically shown for performing spinal reflex conditioning for an anatomical limb 1 (specifically an arm in the illustrative example, although the approach is also suitable for spinal reflex conditioning of a leg) of a person 2. The system includes an armband 10 with stimulation electrodes 11 (or, alternatively, a leg band in the case of conditioning for a leg) that is wearable (and shown as being worn) on the anatomical limb 1. The illustrative stimulation electrodes 11 are arranged on the armband 10 to electrically stimulate a peripheral nerve of the anatomical limb 2 to evoke a spinal reflex. For example, the stimulation electrodes 10 may be transcutaneous electrodes disposed on the armband 10 in contact with the skin of the arm 2 when the armband 10 is worn on the arm 2. (Note that if the armband 10 is opaque and the stimulation electrodes 11 are on an inner surface thereof then the stimulation electrodes 11 would be hidden from view). In other embodiments, implanted electrodes may be employed, or the stimulation electrodes 11 may be implemented as individual stimulation electrodes adhered to the skin by electrically conductive adhesive gel or the like.

The system further includes a sleeve 12 that is wearable on the anatomical limb (and shown worn on the arm 2). The sleeve includes electrodes (not shown) arranged to acquire electromyography (EMG) signals from the anatomical limb 2. For example, the electrodes may be transcutaneous electrodes disposed on an inner surface of the sleeve 12 in contact with the skin of the arm 2 when the sleeve 12 is worn on the arm 2. In some embodiments, the sleeve 12 includes at least 100 electrodes distributed over the surface of the arm 2 when the sleeve is worn on the arm 2. This enables high-density EMG (HD-EMG) measurements, e.g. capable of acquiring EMG signals from a plurality of flexor and extensor muscles in the arm using the sleeve 12.

The system further includes a vagus nerve stimulation (VNS) device. FIG. 1 illustrates two options as suitable VNS devices. In one embodiment, the VNS device comprises a transcutaneous auricular vagus nerve stimulation (taVNS) device 14 that fits over an ear of the person 1 and is powered by a built-in VNS stimulator circuit (e.g. battery-powered) or has a wired connection to an external VNS stimulator (not shown). The taVNS device 14 advantageously provide non-invasive vagus nerve stimulation, and operates on the principle that a branch of the vagus nerve lies close to the surface of the skin in the ear region. In another embodiment, the VNS device comprises an implanted VNS stimulator 15 having lead wires 16 electrically coupled with the vagus nerve in the neck. For example, the distal ends of the lead wires 16 may be wrapped around the vagus nerve at the carotid sheath. The implanted VNS stimulator 15 advantageously provides strong coupling with the vagus nerve, but requires implantation.

The system further includes an electronic spinal reflex conditioning controller 20 that includes an electronic processor 22, a non-transitory storage medium 24 storing instructions readable and executable by the electronic processor 22 to perform spinal reflex conditioning methods as disclosed herein, an armband stimulator 26 connected to (the stimulation electrodes 11 of) the armband 10 to apply electrical stimulation to the arm 2, an EMG readout amplifier 28 connected with the sleeve 12 to read EMG signals using the electrodes of the sleeve 12, and a VNS trigger 30 that wirelessly transmits VNS stimulation trigger signals to the VNS device 14 or 15. For example, the VNS trigger 30 can be embodied as a Bluetooth™ transmitter or other type of short range wireless transmitter that transmits VNS trigger signals to the VNS device 14 or 15 to trigger VNS stimulations. The electronic processor 22 may, for example, comprise an integrated circuit (IC) microprocessor or microcontroller and ancillary electronics such as a memory. The non-transitory storage medium 24 may comprise a flash memory or other electronic memory (which may optionally be onboard the IC electronic processor 22), a hard disk or other magnetic memory, an optical disk or other optical memory, various combinations thereof, or so forth. Optionally, the non-transitory storage medium 24 may also store relevant data, such as baseline spinal reflex data collected as disclosed herein, and/or records of spinal reflex conditioning therapy.

The electronic spinal reflex conditioning controller 20 may employ various combinations of wired and/or wireless communication connections. For example, while the illustrative embodiment employs on-board VNS stimulation power (e.g. on-board battery of the VNS device 14 or 15) with external VNS trigger 30, in another embodiment the VNS stimulator may be integrated into the electronic spinal reflex conditioning controller 20 and connected to the lead wires 16 or to the taVNS device 14 by a wired connection to deliver VNS stimulation. Similarly, while the illustrative EMG readout amplifier 28 is integrated into the electronic spinal reflex conditioning controller 20 and connected by wiring to the sleeve 12, in another embodiment the EMG readout amplifier may be integrated into the sleeve 12 to acquire and digitize the EMG signals which are then wirelessly sent to the electronic spinal reflex conditioning controller 20 by short-range wireless communication. In some embodiments, the system is entirely portable. To this end, the electronic spinal reflex conditioning controller 20 may include one or more batteries 32 to power the electronic spinal reflex conditioning controller 20. It should also be appreciated that the various components of the electronic spinal reflex conditioning controller 20 may be collected in a single unitary housing as diagrammatically shown, or may be physically implemented as separate components interconnected by suitable wiring and/or wireless links. These are merely some non-limiting illustrative implementation options.

The electronic controller 20 is configured to provide spinal reflex conditioning therapy as follows. The electronic controller 20 is configured to: evoke a spinal reflex by electrically stimulating a peripheral nerve of the anatomical limb 2 using the stimulation electrodes 11 of the armband 10 (or of a leg band, in the case of leg spasticity conditioning); measure the spinal reflex using the sleeve 12 (e.g., by measuring EMG signals corresponding to spasticity of the spinal reflex); and perform VNS using the VNS device 14 or 15 in response to the measured spinal reflex satisfying a positive reinforcement criterion.

The illustrative spinal reflex conditioning therapy has numerous advantages. The positive reinforcement is provided by way of VNS, rather than by an approach such as visual reinforcement that involves higher cognitive centers detached from the nonvolitional physiology of hyperreflexia (or hyporeflexia). By contrast, VNS is believed to provide positive reinforcement by way of noncognitive electrochemical mechanisms such as release of neuromodulators in response to VNS that reinforces physiological activities occurring concurrently with the VNS.

The choice of the positive reinforcement criterion depends on the type of spinal reflex conditioning being performed. In the case of a stroke or SCI patient suffering from spasticity, the appropriate conditioning is hyperreflexia treatment, in which the positive reinforcement criterion suitably comprises the measured spinal reflex being less than a baseline spinal reflex. In other words, the VNS positive reinforcement is provided when the measured spinal reflex is lower than the baseline spinal reflex for that person, so that the VNS is reinforcing a reduction in the spinal reflex. On the other hand, if the measured spinal reflex is higher than the baseline spinal reflex then no VNS is performed, so that this hyperreflexia is not reinforced.

Conversely, if the person is suffering from hyporeflexia (e.g., a patient with muscle weakness in the legs or other anatomy), then the appropriate conditioning is hyporeflexia treatment, in which the positive reinforcement criterion suitably comprises the measured spinal reflex being greater than a baseline spinal reflex. In other words, the VNS positive reinforcement is provided when the measured spinal reflex is higher than the baseline spinal reflex for that person, so that the VNS is reinforcing increased spinal reflex. On the other hand, if the measured spinal reflex is lower than the baseline spinal reflex then no VNS is performed, so that this hyporeflexia is not reinforced.

Advantageously, the spinal reflex conditioning can be performed while the person is ambulatory (e.g., not bedridden). This is facilitated by employing battery-powered devices (e.g., with the controller powered by a battery or batteries 32 and the VNS device 14 or 15 also having an on-board battery). Additionally, it will be appreciated that the spinal reflex conditioning method can be performed without volitional input from the person, since the positive reinforcement is in the form of VNS rather than, for example, visual reinforcement which requires the person 1 to volitionally respond by looking at the display providing the visual reinforcement. This means that the disclosed spinal reflex conditioning can be performed “on the go”, while the person 1 is engaged in other activities, such as manual dexterity rehabilitation therapy performed in FIG. 1 by the person 1 placing an object (not shown) into a target 34 as a nonlimiting illustrative example. While the disclosed spinal reflex therapy can be performed “on the go” without volitional input from the person 1, it will be appreciated that the evocation of a spinal reflex may adversely impact motor control, e.g. by inducing spasticity events. Hence, in some embodiments the electronic spinal reflex conditioning controller 20 may include an on/off switch (not shown) or the like to enable the person 1 to select when the conditioning may be provided.

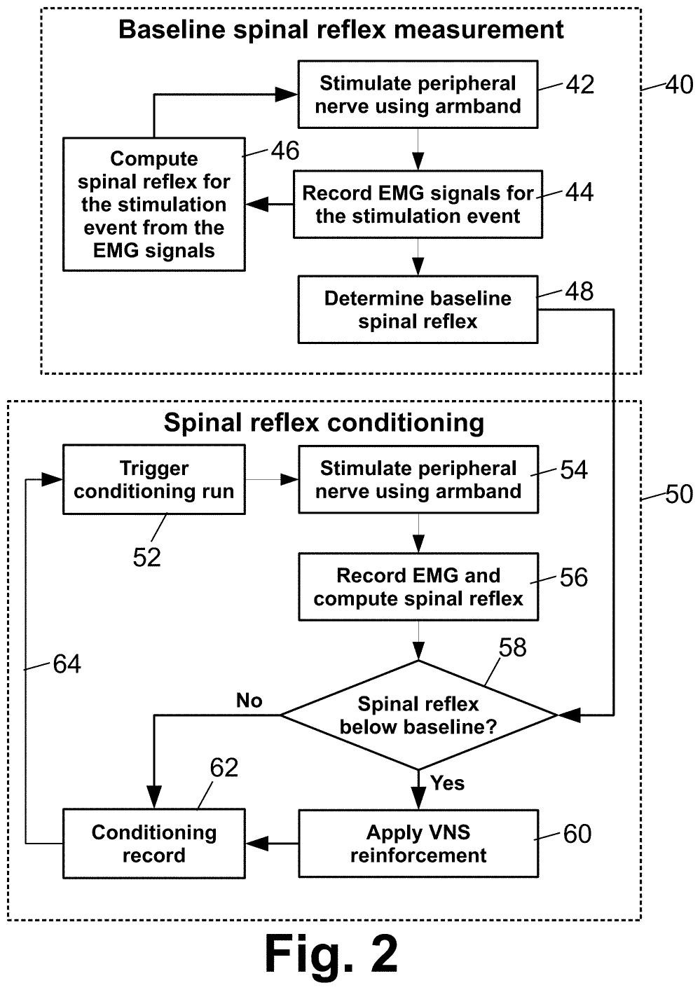

With reference to FIG. 2, a spinal reflex conditioning method suitably performed using the system of FIG. 1 or a similar system is described. The conditioning includes (or, in another view, is preceded by) a baseline spinal reflex measurement 40. This includes stimulating the peripheral nerve using the stimulation electrodes 11 of the armband 10 in an operation 42 and recording EMG signals caused by the resulting spinal reflex in an operation 44 using (the electrodes of) the sleeve 12. The peripheral nerve stimulation 42 should be of sufficient magnitude to evoke the spinal reflex to be conditioned. The appropriate stimulation intensity can be determined empirically during setup of the system. Where the anatomical limb being conditioned is an arm (as illustrated in FIG. 1), the spinal reflex may for example be evoked by electrically stimulating one or more of a median peripheral nerve of the arm, a radial peripheral nerve of the arm, and/or an ulnar peripheral nerve of the arm. The choice of which nerve or nerves to stimulate can be determined by the placement of the stimulation electrodes 11 on the armband 10 and its placement on the arm 2. For example, the armband 10 can optionally include a positioning marker that is to be aligned with the biceps of the arm.

In an operation 46, a measure of the spinal reflex is computed from the EMG signals acquired at the operation 44. This process 42, 44, 46 is repeated as indicated by the arrow feeding back to block 42 for some number of times to collect baseline spinal reflex data for a number of calibration runs with a stimulation event 42 starting each calibration run.

In the operation 46, the spinal reflex measure is suitably computed from the EMG signals acquired at operation 44 for each peripheral nerve stimulation event 42. For example, in one approach the average EMG intensity over all electrodes of the sleeve 12 at each sample time is computed, and the spinal reflex measure is taken as the largest average EMG intensity observed for any time sample over the sampling time interval. The sampling time interval can extend from a fixed time after the stimulation 42 ends to a time sufficient to ensure that any spinal reflex will have occurred and dissipated.

In another approach for computing the spinal reflex measure at the operation 46, the maximum EMG intensity over all electrodes of the sleeve 12 at each sample time is computed, and the spinal reflex measure is taken as the largest maximum EMG intensity observed for any time sample over the sampling time interval. Since the maximum EMG intensity can be sensitive to noise, various smoothing and/or outlier removal approaches can be employed to suppress the effects of noise in determining the maximum EMG intensity at a given time sample. For example, the “maximum” EMG signal can be the average of the N largest EMG signals measured at that time interval (where N>1 and is suitably chosen to balance noise suppression versus artificially depressing the calculated maximum EMG intensity).

In another approach for computing the spinal reflex measure at the operation 46, the average EMG intensity is integrated over the electrodes of specific spatial regions of the sleeve 12, optionally corresponding to specific muscle groups, and the largest average EMG intensity observed over the spatial regions and the sampling time interval is taken as the measure of the spinal reflex. This approach provides noise suppression by way of the spatial average over each spatial region while also providing for detection of a localized spinal reflex. In some embodiments, the spatial regions may be selected based on the underlying musculature anatomy, e.g. with each spatial region corresponding to a specific muscle or muscle group. Normalization by the spatial region area can be used to accommodate different areas for the spatial regions.

These are merely illustrative examples, and other approaches are contemplated for computing the spinal reflex measure at the operation 46 from the EMG signals acquired at block 44 for each peripheral nerve stimulation event 42.

The loop 42, 44, 46 is performed a number of times, with a delay between each spinal reflex measure computation 46 and the next stimulation event 42 to allow the spinal reflex to dissipate and to avoid overstimulating the arm. The looped operations 42, 44, 46 implement collection of baseline spinal reflex data, for example comprising the spinal reflex measure determined for each stimulation event 42. Thereafter, in an operation 48, the baseline spinal reflex is determined as an average spinal reflex measure, median spinal reflex measure, or other statistical average of the baseline spinal reflex data. Since the goal of the spinal reflex conditioning is to improve on this baseline spinal reflex measure (either by decreasing it in the case of hyperreflexia treatment, or by increasing it in the case of hyporeflexia treatment), the baseline spinal reflex determined at operation 48 may optionally be adjusted either up or down by some predetermined amount (e.g. by 5% or another chosen percentage adjustment) to bias the positive reinforcement. For example, in the case of hyperreflexia treatment where the goal is to reduce the spinal reflex, the true average spinal reflex measure might be decreased by 5% so that only spinal reflexes that are significantly below the average (here, 5% or lower below that true average) are positively reinforced.

The output of the baseline spinal reflex measurement is the baseline spinal reflex output by the operation 48. This is then used as the baseline for deciding whether to administer positive VNS reinforcement during the subsequent spinal reflex conditioning 50. In this conditioning, a conditioning run is triggered at an operation 52. Because the spinal reflex conditioning 50 can be performed while the person is ambulatory, and does not require volitional input from the person 1, a conditioning run may be triggered, for example, at random times or at fixed time intervals, e.g. every 5 minutes for example. Preferably, the operation 52 ensures that there is a suitable rest interval between successive conditioning runs to avoid overstressing the muscles. In some embodiments, the operation 52 optionally may also receive external information to avoid performing a conditioning run at an inopportune time. For example, if the person 1 is engaged in an activity that could be interrupted or adversely affected by evoking a spinal reflex then a sensor detecting this activity can inform the electronic spinal reflex conditioning controller 20 to not trigger any conditioning runs. Similarly, if the person 1 is sleeping then this might be detected by an inertial measurement unit (IMU) such as an accelerometer worn by the person, and the electronic spinal reflex conditioning controller 20 can thus avoid triggering conditioning runs while the person is sleeping (since the evoked spinal reflex can result in awakening the person).

A triggered condition run starts by stimulating the peripheral nerve using the stimulation electrodes 11 of the armband 10 in an operation 54 which is performed in the same way as the calibration stimulation 42, and then recording EMG signals caused by the resulting spinal reflex and computing the spinal reflex measure in an operation 56 corresponding to the calibration operations 44 and 46. In an operation 58, it is determined whether the spinal reflex determined at the operation 56 satisfies a positive reinforcement criterion. In the illustrative example, hyperreflexia treatment is assumed, and the operation 58 thus determines whether the spinal reflex determined at the operation 56 is less than the baseline spinal reflex output by the baseline spinal reflex measurement 40 (and more specifically by the operation 48 thereof). If the positive reinforcement criterion is satisfied (here, if the spinal reflex measurement determined at the operation 56 is less than the baseline spinal reflex) then in an operation 60 VNS is performed using the VNS device 14 or 15 to provide positive reinforcement for the low spinal reflex measured at the operation 56. On the other hand, if the positive reinforcement criterion is not satisfied (here, if the spinal reflex measurement determined at the operation 56 is equal to or greater than the baseline spinal reflex) then the positive VNS reinforcement operation 60 is not performed.

As another example, if hyporeflexia treatment is being performed, then the analog to the operation 58 would suitably determine whether the spinal reflex determined at the operation 56 is greater than the baseline spinal reflex output by the baseline spinal reflex measurement 40 (and more specifically by the operation 48 thereof), and the analog to the operation 60 would provide VNS reinforcement if the spinal reflex determined at the operation 56 is greater than the baseline spinal reflex.

Regardless of whether positive VNS reinforcement is provided in the operation 60, in some embodiments an operation 62 maintains a conditioning record by recording the spinal reflex obtained at the operation 56, optionally along with an indication of whether positive VNS reinforcement was applied.

As indicated by a flowback arrow 64, the conditioning run 52, 54, 56, 58, and conditionally 60 may be repeated at time intervals determined by the triggering 52 to provide continuous spinal reflex conditioning for as long as the system is operating. Advantageously, these conditioning runs can be performed while the person is ambulatory, and does not require volitional input from the person, so that they can be done as the person is engaged in various activities.

In the following, some further aspects and/or illustrative embodiments are described.

H-reflex conditioning is a therapeutic intervention for people with motor dysfunction after spinal cord injury and stroke. This technique involves upper arm stimulation combined with electromyography (EMG) of spastically impaired muscles (primarily forearm flexor muscles) and visual feedback to condition the h-reflex back towards normal. Embodiments disclosed herein integrate the sleeve 12 which measures high density EMG activity across many flexor and extensor muscles in the forearm, many of which are typically affected by spasticity or hyperactive stretch reflexes, and upper arm stimulation via the stimulation electrodes 11 of the armband 10 with visual feedback, e.g., visual feedback displayed on a display, visual feedback provided as augmented reality (AR) content via an AR headset or AR eyeglasses, or so forth. By using the sleeve 12 to measure EMG, multiple muscles in the forearm can be easily targeted and recorded simultaneously to improve spasticity caused by stroke. In one illustrative example, the person 1 dons the sleeve 12 and the armband 10 with the upper arm stimulation electrodes 11 while receiving visual feedback regarding their h-reflex. With each stimulation, a criterion threshold for the h-reflex is set and when the h-reflex response is below the criterion threshold then positive visual feedback is provided, thus down-regulating the h-reflex. (This example is for treating hyperreflexia. For treating hyporeflexia, the positive visual feedback is suitably provided when the h-reflex response is above the criterion threshold.) This process is repeated a number of times during each session in order to slowly decrease the hyperactive stretch reflexes across multiple affected muscles thus improving motor dysfunction. This framework can substantially improve the process as the sleeve 12 provides high-density EMG across many affected muscles in the forearm simultaneously. The visual feedback produces long-lasting improvement of the h-reflex and motor function. Furthermore, along with the improvement of motor function, this process may improve sensorimotor function and more specifically, proprioceptive function which is also typically impaired in stroke survivors. This monosynaptic stretch reflex (h-reflex) directly involves type 2 sensory transmission from nuclear bag sensory fibers from the affected skeletal muscles which transmit information regarding proprioception. Motor and sensory function are often working together and by improving motor function, sensory function may also be improved.

In other embodiments, as illustrated in FIG. 1 adding VNS allows h-reflex conditioning to be done without the user being actively engaged, such as is the case when using visual reinforcement. Additionally, VNS is expected to more effectively drive plasticity in the central nervous system due to the direct activation of central neuromodulatory systems.

In other embodiments, VNS and visual feedback can be combined.

In other embodiments, aural positive feedback can be employed, for example playing music enjoyed by the person.

In yet other embodiments, the sleeve 12 may provide pleasing somatosensation as the positive feedback, by energizing the electrodes of the sleeve 12 to produce the pleasing somatosensation.

Various combinations of the foregoing positive feedback or other types of positive feedback are also contemplated.

Various disclosed approaches for spinal reflex conditioning can advantageously be used to condition multiple muscles simultaneously during a conditioning session.

Various disclosed approaches for spinal reflex conditioning can advantageously measure spasticity in real-time in multiple muscle groups simultaneously.

Various disclosed approaches for spinal reflex conditioning can advantageously provide personalized and algorithmic control over h-reflex criterion.

Various disclosed approaches for spinal reflex conditioning can advantageously be used for other neurological injuries or diseases that affect motor function (spinal cord injury, peripheral nerve injury, traumatic brain injury etc.).

Various disclosed approaches for spinal reflex conditioning advantageously pair VNS with h-reflex conditioning.

Various disclosed approaches for spinal reflex conditioning that combine both visual and VNS positive feedback capability optionally can provide personalized algorithmic control over when h-reflex would be paired with visual feedback and/or VNS.

Various disclosed approaches for spinal reflex conditioning that employ visual positive feedback advantageously can be used in clinic or at home as the therapy is fully automated and only requires the user to follow visual feedback.

Various disclosed approaches for spinal reflex conditioning that employ VNS positive feedback advantageously can be fully automated and do not require the participant's attention.

Various disclosed approaches for spinal reflex conditioning that employ VNS positive feedback can be used with either an implantable VNS stimulator 15 or a transcutaneous auricular vagus nerve stimulation (taVNS) device 14.

The sleeve 12 suitably measures muscle activity in the arm. A grid stimulator placed on the upper arm (for example, implemented as the stimulation electrodes 11 of the armband 10) can target the individual nerves of the forearm (median, ulnar, and/or radial peripheral nerves, for example), and this stimulation is used to evoke the h-reflex. The sleeve 12 can measure the m-wave (motor wave) and also the resulting h-wave from the h-reflex (the reflexive muscle activity in the arm).

For embodiments employing visual feedback, in one suitable approach the visual feedback comprises a displayed representation of the magnitude of the h-wave presented to the user. To up-condition the h-reflex (e.g. to treat hyporeflexia), the person 1 is positively rewarded through visual feedback when the h-wave is greater than the previous 100 trials (or some other set of historical reference runs). Vice versa, to down-condition the h-reflex (e.g. to treat hyperreflexia), the user is positively rewarded through visual feedback when the h-wave is less than the previous 100 trials.

For embodiments employing VNS positive feedback, to up-condition the h-reflex (e.g. to treat hyporeflexia), VNS is delivered if the h-wave is greater than the previous 100 trials (or other set of historical reference runs). To down-condition the h-reflex (e.g. to treat hyperreflexia), VNS is delivered if the h-wave is less than the previous 100 trials.

In some further embodiments, a combination of positive visual reinforcement and VNS is delivered simultaneously.

The preferred embodiments have been illustrated and described. Obviously, modifications and alterations will occur to others upon reading and understanding the preceding detailed description. It is intended that the invention be construed as including all such modifications and alterations insofar as they come within the scope of the appended claims or the equivalents thereof.

1. A method of performing spinal reflex conditioning for an anatomical limb of a person, the method comprising:
evoking a spinal reflex by electrically stimulating a peripheral nerve of the anatomical limb;
measuring the spinal reflex using electromyography (EMG) signals acquired from the anatomical limb; and
performing vagus nerve stimulation (VNS) in response to the measured spinal reflex satisfying a positive reinforcement criterion.

2. The method of claim 1 comprising a hyperreflexia treatment wherein the positive reinforcement criterion comprises the measured spinal reflex being less than a baseline spinal reflex.

3. The method of claim 1 comprising a hyporeflexia treatment wherein the positive reinforcement criterion comprises the measured spinal reflex being greater than a baseline spinal reflex.

4. The method of claim 2 further comprising:
determining the baseline spinal reflex by:
repeatedly evoking the spinal reflex by electrically stimulating the peripheral nerve of the anatomical limb and measuring the spinal reflex using EMG signals acquired from the anatomical limb to generate baseline spinal reflex data, and
determining the baseline spinal reflex as a statistical average of the baseline spinal reflex data.

5. The method of claim 1 wherein the spinal reflex is evoked by electrically stimulating the peripheral nerve of the anatomical limb using stimulation electrodes disposed on an armband or leg band wrapped around an upper portion of the anatomical limb.

6. The method of claim 5 wherein the anatomical limb is an arm and the spinal reflex is evoked by electrically stimulating one or more of a median peripheral nerve of the arm, a radial peripheral nerve of the arm, and/or an ulnar peripheral nerve of the arm.

7. The method of claim 5 wherein the anatomical limb is an arm and the measuring of the spinal reflex using EMG signals acquired from the anatomical limb includes acquiring EMG signals from a plurality of flexor and extensor muscles in the arm using a sleeve worn on the arm and including at least 100 electrodes.

8. The method of claim 1 wherein the VNS is performed using transcutaneous auricular vagus nerve stimulation (taVNS).

9. The method of claim 1 wherein the method is performed while the person is ambulatory.

10. The method of claim 1 wherein the method is performed without volitional input from the person.

11. A system for performing spinal reflex conditioning for an anatomical limb of a person, the system comprising:
stimulation electrodes arranged to electrically stimulate a peripheral nerve of the anatomical limb to evoke a spinal reflex;
a sleeve wearable on the anatomical limb and including electrodes arranged to acquire EMG signals from the anatomical limb;
a vagus nerve stimulation (VNS) device; and
an electronic controller configured to evoke a spinal reflex by electrically stimulating the peripheral nerve of the anatomical limb using the stimulation electrodes, measure the spinal reflex using the sleeve, and perform VNS using the VNS device in response to the measured spinal reflex satisfying a positive reinforcement criterion.

12. The system of claim 11 wherein the electronic controller is configured to perform the VNS using the VNS device in response to one of:
(i) the measured spinal reflex being less than a baseline spinal reflex whereby the system is configured to treat hyperreflexia, or
(ii) the measured spinal reflex being greater than a baseline spinal reflex whereby the system is configured to treat hyporeflexia.

13. The system of claim 11 wherein the VNS device comprises one of:
a transcutaneous auricular vagus nerve stimulation (taVNS) device; or
an implanted VNS stimulator having lead wires electrically coupled with the vagus nerve.

14. The system of claim 11 further comprising:
an armband or leg band, wherein the stimulation electrodes are arranged on an armband or leg band to contact the anatomical limb.

15. The system of claim 11 wherein the system is a battery-powered mobile system configured to provide muscle spasticity conditioning while the person is ambulatory.

16. The system of claim 11 wherein the sleeve includes at least 100 electrodes arranged to acquire spatially resolved EMG signals from the anatomical limb.

17. A non-transitory storage medium storing instructions readable and executable by an electronic processor to perform spinal reflex conditioning for an anatomical limb of a person by operations including:
evoking a spinal reflex by energizing stimulation electrodes to electrically stimulate a peripheral nerve of the anatomical limb;
measuring the spinal reflex using electromyography (EMG) signals acquired from the anatomical limb using electrodes of a sleeve configured to be worn on the limb;
determining whether the measured spinal reflex satisfies a positive reinforcement criterion; and
controlling a vagus nerve stimulation (VNS) device to deliver VNS to a vagus nerve of the person in response to the measured spinal reflex satisfying the reinforcement criterion.

18. The non-transitory storage medium of claim 17 wherein the reinforcement criterion comprises the measured spinal reflex being less than a baseline spinal reflex.

19. The non-transitory storage medium of claim 17 comprising a hyporeflexia treatment wherein the reinforcement criterion comprises the measured spinal reflex being greater than a baseline spinal reflex.

20. The non-transitory storage medium of claim 18 wherein the instructions are further readable and executable by the electronic processor to:
generating baseline spinal reflex data by repeatedly evoking the spinal reflex by controlling the energizing the stimulation electrodes to electrically stimulate the peripheral nerve of the anatomical limb and measuring the spinal reflex using EMG signals acquired from the anatomical limb using the electrodes of the sleeve; and
determining the baseline spinal reflex as a statistical average of the baseline spinal reflex data.
